(19) United States
(12) Patent Application Publication
(10) Pub. No.: US 2022/0280275 A1
(43) Pub. Date: Sep. 8, 2022

(54) MEDICAL KIT AND MEDICAL SYSTEM FOR THE TREATMENT OF ANEURYSMS

(71) Applicant: Acandis GmbH, Pforzheim (DE)

(72) Inventors: Michael Büchert, Pforzheim (DE); Eugen Schönberger, Pforzheim (DE); Giorgio Cattaneo, Karlsruhe (DE)

(21) Appl. No.: 17/632,611

(22) PCT Filed: Jul. 21, 2020
(86) PCT No.: PCT/EP2020/070499

§ 371 Date: Feb. 3, 2022

(30) Foreign Application Priority Data
Aug. 9, 2019 (DE) 10 2019 121 546.3

(51) Int. Cl. A61F 2/01; A61B 17/12

(52) U.S. Cl. CPC A61F 2/013; A61F 2/0105; A61F 2/011; A61B 17/1214; A61B 17/12181; A61B 17/12113; A61F 2002/016; A61B 2017/1205

(57) ABSTRACT

The invention relates to a medical kit for the treatment of aneurysms, including a main catheter and a cover device for temporarily covering as aneurysm, which cover device can be moved through the main catheter to a place of treatment. The cover device includes a self-expanding lattice structure that has a cylindrical portion, open at a distal end and at least partially provided with a cover, and a funnel-type portion that is permanently connected to a transport wire, movable inside the main catheter, and a total lateral area of which is not covered so that when the lattice structure is expanded, blood can flow through it in the direction of its longitudinal axis. The invention further relates to a medical system.

The invention relates to a medical set for the treatment of aneurysms in accordance with the preamble of patent claim 1. Furthermore, the invention relates to a medical system.

WO 2014/177634 A1 describes a highly flexible stent, which has a compressible and expandable mesh structure, wherein the mesh structure is formed in one piece. The mesh structure comprises closed cells which are each delimited by four mesh elements. The mesh structure has at least one cell ring which comprises between three and six cells.

Furthermore, to the Applicant's knowledge, stents with mesh structures are known that are formed from a single wire.

The wire is braided with itself in order to form a tubular network. At the axial ends of the tubular network, the wire is curved round so that atraumatically acting loops are formed. The axial ends may flare outwards in a funnel shape.

The known medical device is particularly suitable for the treatment of aneurysms in small cerebral blood vessels. Blood vessels of this type have a very small cross sectional diameter and are often highly tortuous. For this reason, the known stent is highly flexible in configuration, so that on the one hand it can be compressed to a very small cross sectional diameter and on the other hand it has a high bending flexibility, which enables it to be delivered to small cerebral blood vessels.

For the treatment of aneurysms in cerebral blood vessels, it is advantageous to use stents that span an aneurysm and screen it from the flow of blood inside the blood vessel. To enable this, providing stents with a covering is known; it occludes the cells of the stent and thus prevents the flow of blood into an aneurysm.

A further supplementary or alternative method for the treatment of aneurysms is the implantation of so-called coils into the aneurysm, which, once there, lead to blood clotting. The resulting thrombus then prevents blood from circulating in the aneurysm and thus removes the risk of a rupture with accompanying haemorrhage.

However, especially in broad-necked aneurysms, the coils have a tendency to displace during implantation into the blood vessel and thus cause the main vessel lumen to become occluded. In the “Balloon Assisted Coiling” technique, catheters are positioned in the vessel with “Compliance Balloons”, in particular at the level of the neck of the aneurysm. The balloon, which is filled with contrast agent, then occludes the neck of the aneurysm during placement of the coil and forces the coil into a compact arrangement inside the aneurysm sac. Because coils are plastically deformable, they then remain in their shape. They do not come out of the aneurysm even when the balloon is removed. However, a problem arises in this regard, in particular because of the fact that the balloon occludes the vessel. In a lengthy procedure (in the case of large aneurysms, a plurality of coils are positioned, and the procedure can last several minutes), the flow of blood is completely cut off during that time. Even though collateral vessels supply neighbouring tissue, the risk of underperfusion remains.

Furthermore, during the procedure a catheter through which the coils are fed is “jailed”, i.e. wedged at the side of the balloon. In the event that the coil catheter has to be changed (for example in the case of damage) when the procedure has not yet been completed, the balloon has to be deflated (emptied) in order to be able to withdraw the catheter. In this phase, coils that are not yet fully compacted in the vessel could become displaced. This could result in occlusion of the vessel.

In the light of the foregoing, the objective of the invention is to provide a medical set for the treatment of aneurysms with the aid of which the risk of vessel occlusion is at least reduced. A further objective of the invention is to provide a medical system.

In accordance with the invention, this objective is achieved in respect of the medical set by the subject matter of patent claim 1, and in respect of the medical system by the subject matter of patent claim 15.

The subject matter of the dependent claims pertains to preferred embodiments, further embodiments and variations. Specifically, the objective is achieved by means of a medical set for the treatment of aneurysms, having a main catheter as well as having a covering device, which is moveable through the main catheter to a treatment site. The term “treatment site” as used here can be understood to mean a position along a vessel at which the aneurysm is formed. In this respect, the covering device serves as a temporary covering for the aneurysm, wherein the covering device comprises a self-expandable mesh structure.

The mesh structure has a cylindrical section, which is open at a distal longitudinal end and at least part of which is provided with a covering. In this regard, the term “longitudinal end” can be understood to mean the end of the cylindrical section which faces away from the main catheter. Furthermore, the mesh structure has a funnel-shaped section, which is permanently connected to a transport wire displaceable inside the main catheter and which is free from a covering over an entire circumferential surface. The term “circumferential surface” as used herein can be understood to mean a generatrix of the funnel-shaped section, so that by means of this configuration, which is free from a covering, in an expanded state, the mesh structure is perfusible by blood in the longitudinal axial direction. The term “funnel-shaped section” as used herein can be understood to mean a rotationally symmetrical funnel-shaped section, but also alternatively, a non-rotationally symmetrical funnel-shaped section, for example in the form of a non-concentric connection point. The non-concentric connection point in this regard may, for example, be disposed on the generatrix. As another alternative, the funnel-shaped section may be formed solely by individual wires, preferably by two to six wires and especially by exactly two wires of the mesh structure, which spread out from loops or from the last cells of the mesh structure. Thus, in this case, a closed mesh structure is formed. In this case, attachment of the mesh structure to a transport wire may also be carried out on the circumferential side at an edge of the mesh structure in which, for example, one or more wires form an extension or are extended which is connected to the transport wire. A configuration of this type is to be seen, for example, in the Applicant's published document DE 10 2009 056 450 A1, reference to which should be made in this regard. In this manner, full perfusibility of the mesh structure is obtained because even the attachment to the transport wire is disposed substantially parallel to the vessel wall and along the vessel wall of the vessel.

Furthermore, the funnel-shaped section may also have a stepped tapered contour. The term “perfusible by blood in the longitudinal direction” as used herein can be understood to mean that the flow of blood passing through the vessel is not substantially perturbed by the configuration of the covering device.

Appropriately, the mesh structure includes a shape memory alloy, in particular nitinol, or is preferably formed from a material of this type. Advantageously, the shape memory alloy is laser cut in order to produce the mesh structure.

The advantage here is that by means of the perfusibility of the mesh structure by blood in the longitudinal direction, a flow of blood in the vessel is guaranteed during placement of a coil. Because of the longitudinally axially perfusible mesh structure, then advantageously, a flow of blood in the longitudinal direction through the blood vessel is barely hindered, but a flow of the blood into a branching aneurysm is prohibited by the covering, or at least ingress of a flow into the aneurysm is reduced. A possible and unwanted occlusion of the vessel, as can occur, for example, with the aforementioned technique using a balloon, is therefore at least reduced and preferably excluded.

In one embodiment, the covering is configured as an electrospun covering.

In an electrospun fabric, pores are usually irregular in shape. In any case, the production method does not permit pores to be produced with a specific arrangement or shape. However, the pore size can be adjusted with the aid of the process parameters at least to the extent that it is ensured that at least a portion of the pores have a certain minimum size.

As an example, the electrospinning process may be carried out directly on the mesh structure, so that a bond with the mesh structure is produced at the same time as the covering is formed. The covering may be connected to the mesh structure in a materially bonded manner. As an example, the covering may be bonded to the mesh structure by means of an adhesive bond. The adhesive bond may be produced by means of a bonding agent. The bonding agent may, for example, comprise or consist of polyurethane.

Furthermore, the covering produced from an electrospun fabric is extremely thin and flexible, which compromises the flexibility of the mesh structure to only a little or to no extent. In particular, the covering barely inhibits compression of the mesh structure, which is in contrast to prior art coverings, which are produced from textile materials. Overall, then, the entire covering device can be compressed to a considerably smaller cross sectional diameter, and thus can be fed via small catheters into particularly small blood vessels. This is of particular relevance for the treatment of aneurysms in cerebral blood vessels; the invention is particularly suitable for this purpose.

Thus, treatments are also possible using the medical set in accordance with the invention in blood vessels that could not be accessed with medical devices of the prior art, which have a mesh structure and a covering. Because of the high compressibility of the device in accordance with the invention, very low delivery forces are generated when delivering through a catheter. In particular, the delivery forces in the case of the device with the covering may be the same as or smaller than delivering the mesh structure alone.

Furthermore, by means of the covering device, which is appropriately disposed at the treatment site, i.e. at the level of the aneurysm, escape of the coil from the aneurysm during and after placement of the coil can be prevented, so that the risk of occlusion of the vessel caused by the coils may also be at least greatly reduced, and preferably excluded.

In order to guarantee a sufficient flexibility for the covering, it is preferably formed from irregular filaments arranged in the form of a network which have a filament thickness of between 0.1 μm and 3 μm, in particular between 0.2 μm and 2 μm, in particular between 0.5 μm and 1.5 μm, in particular between 0.8 μm and 1.2 μm.

In one embodiment, the covering is porous and in particular can be perfused by blood. The term “porous” as used here can be understood to mean that the covering is in the form of a network or is configured as a network. The concept behind this embodiment is to be able to supply cells in the region of the covering with blood and therefore with the aforementioned nutrients, so that no supply depletion occurs during placement of the coils. Furthermore, the concept behind this embodiment is that covering the aneurysm at least reduces the flow rate of the blood and thus at least reduces pulsation of the blood which flows through the vessel, because this pulsation could make placement of the coils more difficult.

The medical set in accordance with the invention can therefore cover an aneurysm properly in order to retain the coil placed in the aneurysm, but at the same time allows nutrients to be supplied to the aneurysm. In addition, the supply of nutrients to branched blood vessels and adjacent vessel inner walls is obtained by means of the medical device. The covering, which is formed by an electrospun fabric, enables an aneurysm to be covered, but at the same time allows for a certain permeability for blood. This permeability is advantageous, so that the cells of the aneurysm wall can be supplied with nutrients. In this manner, degeneration of the cells and the risk of a possible rupture of the aneurysm is avoided.

In accordance with an alternative embodiment, the covering in particular has a negligible porosity or is impermeable and therefore impermeable to blood. In particular, despite the negligible porosity, which results from the electrospinning process, the covering can be made so impermeable that blood flows past it, and therefore ingress of blood into the aneurysm is prevented. In this regard, the blood preferably flows on the covering of the aneurysm.

Appropriately, the covering has a porosity of at most 70%, and in particular at most 50%. This means that the stability of the covering is on the one hand increased in respect of the force of the coils which, for example, push on the covering when in the deployed state in the aneurysm.

On the other hand, the stability of the covering is advantageously optimized having regard to the fracture strength. In addition, a negligible porosity covering of this type results in a greater, in particular temporary slowing of the blood flow in the region of the covering, which is advantageous during placement of the coils. This means that the aneurysm pulsates less during placement of the coils and therefore permits faster and more accurate placement of the coils.

In one embodiment, the covering has a porosity of at least 5%, in particular of at least 10%, in particular of at least 20%, in particular of at least 30%, in particular of at least 40% and in particular of at least 45%. The concept behind this embodiment is that the porosity obtained with the aforementioned percentages—as already mentioned above—means that a supply to side vessels during placement of the coils, for example, is guaranteed.

In addition, because of its porosity, the covering is especially suitable for microcatheters because the covering is compressible and therefore can be advanced through the microcatheter to the treatment site and also retracted therefrom with low frictional forces. The term “microcatheter” as used here can be understood to mean a catheter which has a diameter in the range from 0.3 mm to 0.75 mm.

In accordance with an advantageous embodiment, the covering extends over the entire circumferential surface of the cylindrical section. In this manner, the aforementioned advantages, in particular as regards a stability of the covering and therefore of the mesh structure, are advantageously optimized.

In accordance with an advantageous embodiment, the covering extends over a portion, in particular over at most 50%, in particular over at most 40% and in particular over at most 30% of the circumference of the cylindrical section. Preferably, then, the covering therefore extends over the treatment site alone, i.e. over an opening of the aneurysm, for example. This further embodiment ensures that on the one hand, an opening of the aneurysm is sufficiently occluded in order to fix the coils placed in it. On the other hand, this ensures that in particular, cells that are at the level of the aneurysm and/or side vessels can be better supplied and further supplied with blood and therefore with nutrients because of the lack of a covering.

In accordance with a supplemental or alternative embodiment, the covering extends over at least 80%, in particular over at least 90% and in particular over 100% of the length of the cylindrical section. This embodiment is particularly suitable for fusiform or broad-neck aneurysms in which a longer section has to be covered. The term “fusiform aneurysms” as used here can be understood to mean aneurysms which extend over at least 50%, in particular over at least 75% of the entire circumference or over the entire circumference of a blood vessel.

In one embodiment, the covering extends over at most 80%, in particular over at most 60%, in particular over at most 40% of the length of the cylindrical section, wherein the covering is distanced from the distal end of the cylindrical section. Because the cylindrical region of the mesh structure is not completely covered, any vessels in the vicinity of the aneurysm and in particular in the vicinity of the opening of the aneurysm are still perfused, i.e. supplied with blood. This embodiment has been shown to be particularly suitable in the case of aneurysms with neighbouring side branches, as well as in the case of smaller aneurysms.

In one embodiment, the covering has at least 10 pores with a size of at least 15 μm² over an area of 100,000 μm². During production of the covering, this minimum size of the pores is in particular set by the duration of the electrospinning process. This combination of a specific minimum number of pores and a minimum size for these pores has in practice been shown to be particularly necessary for a sufficient perfusion of the covering by blood, for identical coverage.

Preferably, the covering is formed by a synthetic material, in particular by a polymer, and preferably by polyurethane. Materials of this type are particularly light and can be readily produced in fine filaments by means of an electrospinning process. On the one hand, the synthetic material can therefore be used to produce a particularly thin and fine-pored covering. On the other hand, the synthetic material already has a high intrinsic flexibility, so that a high compressibility of the medical set is obtained. Alternatively, the covering may also be produced from polyethylene or fluoropolymers or from thermoplastic polyurethanes based on polycarbonate, for example. Furthermore, for example as an alternative or in a supplementary manner, fillers such as anti-thrombogenic substances, for example, may be embedded in the aforementioned materials of the covering before the covering is formed with them by means of the electrospinning process. As an alternative or in a supplementary manner, the covering is coated with substances of this type, for example with antithrombogenic substances. To this end, the surface of the covering is in particular provided with a nanocoating.

In a further embodiment, the covering is disposed on an outside and/or on an inside of the mesh structure. In a situation in which the mesh structure is disposed on the outside of the mesh structure, the mesh structure forms a support structure which applies a sufficient radial force to secure the covering against a vessel wall. In this respect, the mesh structure supports the outwardly disposed covering.

As an alternative or in addition, the covering may be disposed on an inside of the mesh structure. In particular, it is possible for the mesh structure to be embedded between two coverings which are respectively formed by an electrospun fabric. The mesh elements of the mesh structure can therefore be completely surrounded by the electrospun fabric. Specifically, the electrospun fabric of a covering may extend on the inside of the mesh structure through the cells of the mesh structure and be bonded to the electrospun fabric of a covering on the outside of the mesh structure. The mesh elements which delimit the cells are therefore surrounded on all sides by electrospun fabric.

In accordance with a preferred embodiment, the mesh structure is formed from webs, which are connected together in one piece, i.e. they are monolithic, and delimit closed, in particular diamond-shaped cells. Preferably, in the circumferential direction, the mesh structure has 3 to 9, in particular 4 to 6 consecutively disposed cells which form a circumferential cell ring.

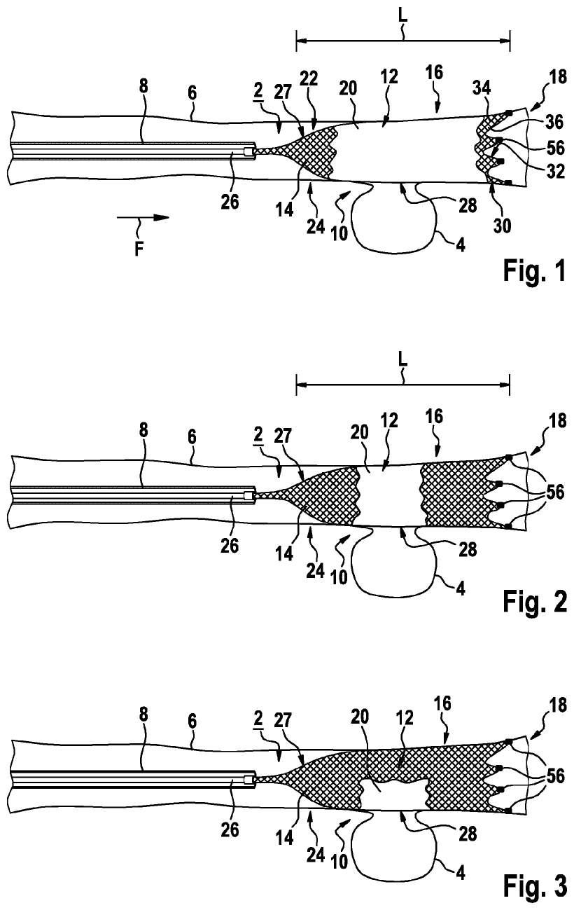

In general, the mesh structure may be configured as a single-piece mesh structure. It is also possible for the mesh structure to be formed from wires which are braided together. The braided wire may consist of a single wire which curves around at the longitudinal ends of the network structure and returns. The wire may be braided with itself in order to form the network structure. The network structure may consist of a plurality of wires which are braided together. The plurality of wires may be curved and returned at one axial longitudinal end while the opposite axial longitudinal end may have wire ends which are open. It is also possible for the interwoven wires to have open wire ends at both axial longitudinal ends. In this respect, in preferred embodiments, the mesh elements form webs, which are coupled together by web connections into one piece (one-piece mesh structure). The wire may have a radiopaque core material and a sheath material produced from a shape memory alloy. In particular, the volume ratio between the core material, preferably platinum, and the volume of the whole of the composite wire is between 20% and 40%, in particular between 25% and 35%. While a braided mesh structure is characterized by a particularly high flexibility, in particular bending flexibility, a one-piece mesh structure has comparatively thin walls, so that the mesh structure influences the blood flow inside a blood vessel to a lesser extent. More preferably, the webs have a thickness with a value in the range between 30 μm and 60 μm. Furthermore, the cells are delimited by a total of four webs, wherein the basic geometry of the cells is substantially diamond-shaped in the preferred design. In particular, each cell is delimited by two pairs of webs, wherein the webs, which are essentially parallel to each other or opposite each other and are not directly connected to each other, form a web pair. A design of this type has already been described in FIG. 1 and in paragraphs [0041] to [0046] of the Applicant's patent document DE 2011 009 371 B3, reference to which should be made in this regard. The webs of a first web pair have a smaller web width than the webs of a second web pair. This arrangement of the webs with different web widths increases the flexibility of the network structure and therefore facilitates introduction of the medical device into human vessels, in particular when these vessels are highly tortuous. The increased flexibility improves attachment to the vessel wall and therefore prevents the formation of congestive regions which stimulates thromboses. The good flexibility and the resulting ease of feeding in and/or through the catheter is particularly important in combination with a biological coating, preferably of fibrin, advantageously fibrin, preferably fibrin including heparin.

In an ancillary aspect, the invention concerns a medical system for the treatment of aneurysms with a medical set. In particular, the set is the medical set described above, which has a main catheter and a covering device, which is moveable through the main catheter to a treatment site for temporary covering of an aneurysm.

In addition, the medical system has at least one embolization means for placement in the aneurysm.

Appropriately, an additional catheter is provided, which has a proximal region, a central region as well as a distal region. The additional catheter is preferably a part of the medical system. The additional catheter acts to deliver the embolization means into the aneurysm. In this regard, the additional catheter is independent of the main catheter and/or is relatively movable with respect to the main catheter.

In accordance with a first embodiment of the medical system, the central region of the additional catheter is disposed inside the covering device. The distal region in this case is disposed outside the covering device and in particular in the aneurysm. The proximal region in this case is disposed parallel to the main catheter. In other words, the additional catheter in this embodiment for the placement of the embolization means is initially fed parallel to the main catheter and then through the covering device, i.e. fed through the covering and into the aneurysm in order to place the embolization means in it.

In accordance with an alternative embodiment of the medical system, the proximal region is disposed parallel to the main catheter. Furthermore, the central region in this case, like the distal region, is disposed outside the covering device, wherein the distal end is still in particular disposed in the aneurysm. In other words, the entire additional catheter is fed substantially parallel to and alongside the main catheter as well as the covering device into the aneurysm. The additional catheter therefore pushes itself under the covering into the aneurysm in order to place the embolization means in it.

Appropriately, in the expanded state, the cells of the mesh structure have an inscribed circle diameter or can be expanded to an inscribed circle diameter which at least corresponds to the external diameter of the additional catheter. The inscribed circle diameter is the diameter of the largest possible circle that can be inscribed in the pores. In other words, the inscribed circle diameter of the pore corresponds to the external diameter of a cylinder that can just be pushed through the pore. In this manner, in particular in the case of the first embodiment of the medical system described above, the additional catheter and especially its central region can easily be introduced into the covering device.

Preferably, the embolization means is formed by a plastically deformable wire, in particular by a coil as mentioned above, or by a liquid, for example by a hydrogel. Embodiments of the embolization means of this type have been shown to be particularly suitable as regards treating aneurysms.

The advantages and preferred embodiments of the medical set apply mutatis mutandis to the medical system, and vice versa. All of the dimensional information pertaining to the medical set and pertaining to the medical system is applicable to an expanded state of the mesh structure, unless indicated otherwise.

Exemplary embodiments of the invention will now be described with the aid of the accompanying diagrammatic drawings. In the highly simplified drawings:

FIG. 1 shows a side view of a medical set in accordance with the invention in a first embodiment,

FIG. 2 shows a side view of a medical set in accordance with the invention in a second embodiment,

FIG. 3 shows a side view of a medical set in accordance with the invention in a third embodiment,

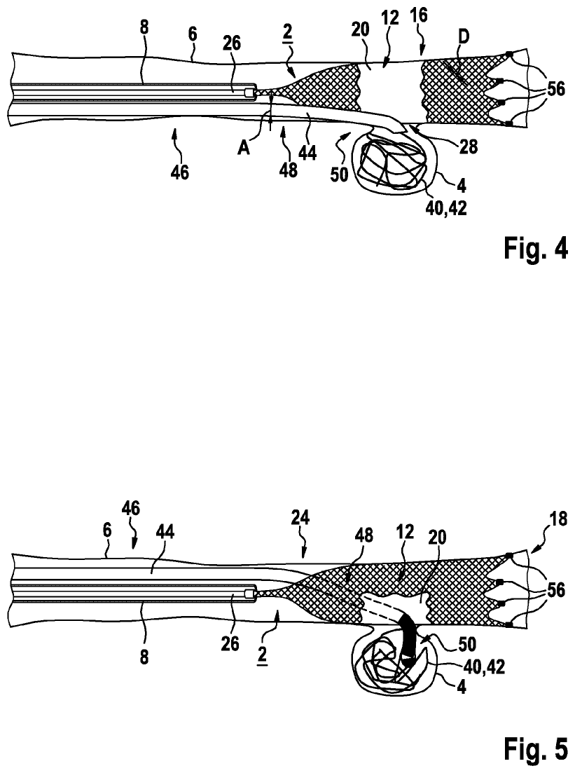

FIG. 4 shows a side view of a medical system in accordance with the invention in a first embodiment,

FIG. 5 shows a side view of a medical system in accordance with the invention in a second embodiment, and

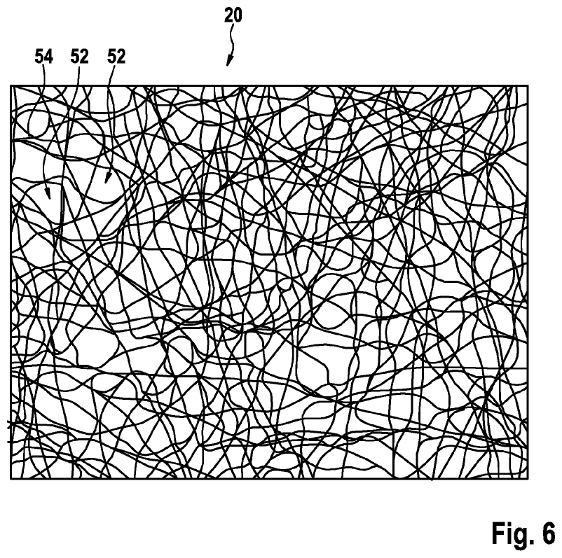

FIG. 6 shows a scanning electron microscope image of a covering of a covering device of a set in accordance with the invention, in a preferred embodiment.

In the figures, parts with identical actions are always designated by the same reference numerals.

The medical set 2 shown in FIG. 1 is used for the treatment of aneurysms 4 and is shown in FIG. 1 in a state disposed inside a vessel 6.

The medical set 2 has a main catheter 8 as well as a covering device 12, which can is moveable through the main catheter 8 to a treatment site 10. The treatment site 10 is preferably a site along the length of the vessel 6 at which the aneurysm 4 is located. The covering device 12 is used for temporarily covering the aneurysm 4, wherein the covering device 12 comprises a self-expandable mesh structure 14. The mesh structure 14 is preferably produced from a shape memory material. Furthermore, the mesh structure 14 has a cylindrical section 16. The cylindrical section 16 is open in configuration at a distal longitudinal end 18. Furthermore, at least a portion of the cylindrical section 16 is provided with a covering 20.

FIG. 1 shows a first embodiment of the medical set 2, in which the covering 20 in particular extends over at least 90% and in particular over 100% of the length L of the cylindrical section 16. In addition, in the exemplary embodiment in accordance with FIG. 1, the covering 20 extends over the entire circumference or the entire circumferential surface of the cylindrical section 16.

Furthermore, the mesh structure 14 has a funnel-shaped section 22, which is formed at a proximal longitudinal end 24 of the mesh structure 14, i.e. in the direction of the main catheter 8.

Furthermore, the funnel-shaped section 22 and therefore the mesh structure 14 are permanently mechanically connected to a transport wire 26 displaceable inside the main catheter 8. By means of this transport wire 26, in a non-expanded state, the mesh structure 14 and therefore the covering device 12 can be advanced and retracted through the main catheter 8.

Furthermore, the funnel-shaped section 22 is free from a covering over the entire circumferential surface 27, preferably formed by the mesh structure 14 alone, so that in the expanded state, the mesh structure 14 is perfusible by blood in the longitudinal axial direction, i.e. in and against a direction of flow F.

In particular, the mesh structure 14 is disposed inside the vessel 6 in a manner such that the covering 20 is positioned at a level with an opening 28 of the aneurysm 4 so that the latter is covered by the covering 20, and at the same time, a flow of blood through the vessel 6 is not stopped. The covering 20 in the exemplary embodiment of FIG. 1 is therefore substantially tubular in configuration, in particular in the manner of a hollow cylinder.

In this regard, the covering 20 is preferably porous in configuration and perfusible by blood, so that a supply of nutrients is ensured to the cells covered by the covering 20. Alternatively, however, the covering 20 may also have negligible porosity or be impermeable and therefore fluid-tight, and in particular not perfusible by blood.

In this regard, in particular when an embolization means 40 is being placed (see FIG. 4 or FIG. 5) inside the aneurysm 4, the covering 20 is used so that after placement, the embolization means 40 cannot escape from the aneurysm 4 until the blood inside the aneurysm 4 has been clotted because of the embolization means 40 and therefore the aneurysm 4 has been effectively occluded.

In the exemplary embodiment in accordance with FIG. 1 and in all of the embodiments below, the covering 20 is formed as an electrospun covering 20.

The covering 20 may furthermore be disposed on an outside and/or on an inside 32 of the mesh structure 14. The mesh structure 14 furthermore has webs 34 or is formed from them, wherein the webs 34 are connected together in one piece, i.e. monolithically and delimit closed, in particular diamond-shaped cells 36. When the medical set 2 is placed inside the vessel 6, the blood therefore flows through the cells 36 and is not blocked. As already described, the webs 34 may also have respective web pairs with different web widths in order to increase the flexibility of the mesh structure 14. This configuration of the web pairs with different web widths is not unique to this exemplary embodiment in accordance with FIG. 1, and is therefore not limited thereto. In fact, all of the exemplary embodiments described in the context of this application may have web pairs configured as described above.

FIG. 2 shows a diagrammatic representation of the medical set 2 in accordance with the invention according to a second embodiment.

The second embodiment corresponds to the first embodiment, which is described in FIG. 1, in its essentials and in respect of the main catheter 8, the mesh structure 14 as well as the transport wire 26. What is essential for the second embodiment is that here, the covering 20 extends over only a portion, in particular over at most 80%, in particular over at most 60% and especially particularly over at most 40% of the length L of the cylindrical section 16.

This means that when the covering device 12 is in position, the covering 20 is only within the region of the opening 28 of the aneurysm 4, so that cells and/or side vessels bordering the aneurysm 4 are not covered by the covering 20. In this manner, the cells and/or the side vessels can still be supplied with blood and therefore with nutrients because the mesh structure 14 is formed with cells 36.

FIG. 3 diagrammatically shows a third embodiment of the medical set 2 in accordance with the invention. In similar manner to the description regarding the second exemplary embodiment, in this case only the differences pertaining to the covering 20 will be described, because all of the other embodiments, preferably in their entirety, in particular substantially, correspond to the embodiment in accordance with FIG. 2 or the embodiment in accordance with FIG. 1. This is particularly the case for the configuration of the main catheter 8, the transport wire 26 as well as the configuration of the mesh structure 14.

In accordance with the third embodiment, the covering 20 extends over only a portion of the circumference of the mesh structure 14 and only over a portion of the length L of the mesh structure 14. In particular in this case, the covering 20 extends in particular over at most 50%, in particular over at most 40% or in particular over at most 30% of the circumference of the cylindrical section 16 of the mesh structure 14.

By means of the third embodiment, a supply to cells and/or side vessels adjacent to the aneurysm 4 is furthermore optimized, because here, preferably, only the opening 28 of the aneurysm 4 is covered and also, cells and/or side vessels which are at the same level as the aneurysm 4 can still be supplied with blood and nutrients. In this manner, for example, a side vessel which is disposed opposite to the aneurysm 4 can be supplied with blood.

As can readily be seen in FIGS. 1 to 3, the medical set 2 and in particular the mesh structure 14 is provided with radiographic markers 56. The radiographic markers 56 are disposed at cell tips of the edge cells 36 of the mesh structure 14. Specifically, the radiographic markers 56 are formed as radiopaque sleeves, for example produced from platinum or gold, which are crimped onto the cell tips of the edge cells 36.

Furthermore, because of the expandability of the mesh structure 14 and also because of the porous configuration of the covering 20, the covering device 12 is configured for use with a main catheter 8, which is configured as a microcatheter.

In addition, it should be noted that the embodiments cited above, in particular as regards the extent of the covering along a length L of the mesh structure 14 and as regards the extent of the covering 20 along a circumference of the mesh structure 14 may be configured and combined in any way. Thus, for example, a configuration is also possible in which the covering 20 extends over the entire length L of the mesh structure, but however, only extends over a portion of the circumference of the mesh structure 14.

FIG. 4 shows a diagrammatic view of a medical system in accordance with the invention according to a first embodiment.

In this regard, the medical system comprises the aforementioned medical set 2 with the main catheter 8 as well as the covering device 12, which has the mesh structure 14 and the covering 20. The configuration of the covering 20 corresponds here to the aforementioned second embodiment.

Furthermore, the medical system has an embolization means which, for example, is formed by a plastically deformable wire 42 or by a liquid. The embolization means is placed inside the aneurysm 4 by means of an additional catheter 44, which is also part of the medical system.

The additional catheter 44 has a proximal region 46, a central region 48 as well as a distal region 50.

In order to place the embolization means 40, in accordance with the first embodiment of the medical system, the additional catheter 44 is disposed substantially parallel to, i.e. alongside the medical set 2 inside the vessel 6. In this regard, the additional catheter 44 and in particular the distal region 50 is “pushed” into the aneurysm 4 between a vessel wall and the covering 20 in order to place the embolization means 40 in the form of the plastically deformable wire 42 (also known as a “coil”) in it. During this and also afterwards, the covering 20 prevents the embolization means 40 from escaping from the aneurysm 4, for example because of the flow of blood.

FIG. 5 diagrammatically shows a second embodiment of the medical system. The only difference here is in the disposition of the additional catheter 44 upon placement of the embolization means 40 inside the aneurysm 4 or into the aneurysm 4.

In accordance with the second embodiment, the proximal region 46 of the additional catheter 44 is fed substantially parallel to, i.e. alongside the main catheter 8, while the central region 48 is fed through the covering device 12 and in particular through the mesh structure 14 into the interior of the mesh structure 14. This can in particular happen because the inscribed circle diameter D of the cells 36 of the mesh structure 14 in the expanded state corresponds to at least the external diameter A of the additional catheter 44 (not shown here to scale).

In this way, the additional catheter 44 and in particular the central region 48 can easily be introduced into the mesh structure 14. Furthermore, the distal region 50 passes through and out of the mesh structure 14 and inside the aneurysm 4. In addition, the distal region 50 of the additional catheter 44 is also fed through the covering 20. During and after placement of the embolization means 40 in the aneurysm 4, in the medical system in accordance with the second embodiment as well, the covering 20 prevents escape of the embolization means 40 from the aneurysm 4.

The construction of the covering 20 can easily be seen in the scanning electron microscope image of FIG. 6. This shows that the covering 20 has a plurality of pores 52 of irregular sizes, which are respectively delimited by filaments 54. The electrospinning process forms a plurality of filaments 54, which are orientated in an irregular manner with respect to each other. In this way, the pores 52 are formed. FIG. 6 also shows that the pores 52 have a comparatively small pore size, wherein some pores 52, however, are sufficiently large to ensure blood perfusion, for example. Specifically, four pores 52 in FIG. 6 have been highlighted; they are more than 30 μm² in size. The density of the pores 52 that are more than 30 μm² in size indicates that the covering has at least 10 pores 52 of this type over an area of 100,000 μm².

FIG. 6 also shows that the filaments 54 of the covering cross over each other multiple times. However, a particular feature of the electrospinning process is that in the covering 20, sites are present at which exclusively two, i.e. no more than two, filaments 54 intersect. It is clear from this that the wall thickness of the covering 20 is very small and it is therefore very flexible.

The high flexibility of the covering 20 in combination with the high flexibility of the mesh structure 14 ensures that a covering device 12 can be produced which can be introduced into a (blood) vessel 6 through very small delivery catheters. In particular, delivery catheters may be used which have a size of 6 French, in particular at most 5 French, in particular at most 4 French, in particular at most 3 French, in particular at most 2 French. Specifically, the covering device in accordance with the exemplary embodiments described here may be used in catheters which have an internal diameter of at most 1.6 mm, in particular at most 1.0 mm, in particular at most 0.7 mm, in particular at most 0.4 mm.

In particularly preferred variations, the layer thickness of the covering 20 is at most 10 μm, in particular at most 8 μm, in particular at most 6 μm, in particular at most 4 μm. In this regard, at most 4, in particular at most 3, in particular at most 2 filaments 54 intersect. In general, inside the electrospun structure of the covering 20, points of intersection are provided in which only 2 filaments 54 intersect. Preferably, the mesh structure 10 has a cross sectional diameter of between 2.5 mm and 8 mm, in particular between 4.5 mm and 6 mm.

### LIST OF REFERENCE NUMERALS

2 medical set
4 aneurysm
6 vessel
8 main catheter
10 treatment site
12 covering device
14 mesh structure
16 cylindrical section
18 distal longitudinal end
20 covering
22 funnel-shaped section
24 proximal longitudinal end
26 transport wire
27 circumferential surface
28 opening of aneurysm
30 outside
32 inside
34 webs
36 cell
40 embolization means
42 deformable wire
44 additional catheter
46 proximal region
48 central region
50 distal region
52 pore
54 filaments
56 radiographic marker
A external diameter
D inscribed circle diameter
F direction of flow
L length

1-20. (canceled)

21. A medical set for treatment of an aneurysm comprising:
a main catheter; and
a covering device adapted to move through the main catheter to a treatment site to temporarily cover the aneurysm, the covering device including a self-expandable mesh structure having a cylindrical section that is open at a distal longitudinal end and at least partially provided with a covering, wherein the mesh structure further has a funnel-shaped section permanently connected to a transport wire displaceable inside the main catheter, and wherein the funnel-shaped section is free from the covering over an entire circumferential surface of the funnel-shaped section such that, in an expanded state, the mesh structure is perfusible by blood in a longitudinal axial direction of the mesh structure.

22. The medical set according to claim 21 wherein the covering is configured as an electrospun covering.

23. The medical set according to claim 21 wherein the covering is porous.

24. The medical set according to claim 21 wherein the covering has a negligible porosity that is impermeable to blood.

25. The medical set according to claim 21 wherein the covering has a porosity of at most 70%.

26. The medical set according to claim 21 wherein the covering has a porosity of at least 5%.

27. The medical set according to claim 21 wherein the covering extends over an entire circumference of the cylindrical section.

28. The medical set according to claim 21 wherein the covering extends over a portion of at most 50% of a circumference of the cylindrical section.

29. The medical set according to claim 21 wherein the covering extends over at least 80% of a length of the cylindrical section.

30. The medical set according to claim 21 wherein the covering extends over at most 80% of a length of the cylindrical section and wherein the covering is distanced from the distal longitudinal end of the cylindrical section.

31. The medical set according to claim 21 wherein the covering has at least 10 pores with a size of at least 15μm² over an area of 100,000 μm².

32. The medical set according to claim 21 wherein the covering is formed by a synthetic material.

33. The medical set according to claim 21 wherein the covering is disposed on one of an outside or an inside of the mesh structure.

34. The medical set according to claim 21 wherein the mesh structure is formed from webs which are connected together in one piece and which delimit closed cells.

35. A medical system for treatment of an aneurysm comprising:
a main catheter;
a covering device adapted to move through the main catheter to a treatment site to temporarily cover the aneurysm, the covering device including a self-expandable mesh structure having a cylindrical section that is open at a distal longitudinal end and at least partially provided with a covering, wherein the mesh structure further has a funnel-shaped section permanently connected to a transport wire displaceable inside the main catheter, and wherein the funnel-shaped section is free from the covering over an entire circumferential surface of the funnel-shaped section such that, in an expanded state, the mesh structure is perfusible by blood in a longitudinal axial direction of the mesh structure; and
at least one embolization means for placement in the aneurysm.

36. The medical system according to claim 35 further comprising:
an additional catheter having a proximal region, a central region and a distal region, the additional catheter configured to deliver the embolization means into the aneurysm, wherein the additional catheter is one of independent of the main catheter or relatively movable with respect to the main catheter.

37. The medical system according to claim 36 wherein the central region of the additional catheter is disposed inside the covering device and the distal region of the additional catheter is disposed outside the covering device, and wherein the proximal region of the additional catheter is disposed parallel to the main catheter.

38. The medical system according to claim 36 wherein the proximal region of the additional catheter is disposed parallel to the main catheter and wherein the central and distal regions of the additional catheter are disposed outside of the covering device.

39. The medical system according to claim 36 wherein, in the expanded state, cells of the mesh structure one of (i) have an inscribed circle diameter or (ii) are expanded to the inscribed circle diameter, wherein the inscribed circle diameter at least corresponds to an external diameter of the additional catheter.

40. A medical system for treatment of an aneurysm comprising:
a main catheter;
a covering device adapted to move through the main catheter to a treatment site to temporarily cover the aneurysm, the covering device including a self-expandable mesh structure having a cylindrical section that is open at a distal longitudinal end and at least partially provided with a covering, wherein the mesh structure further has a funnel-shaped section permanently connected to a transport wire displaceable inside the main catheter, and wherein the funnel-shaped section is free from the covering over an entire circumferential surface of the funnel-shaped section such that, in an expanded state, the mesh structure is perfusible by blood in a longitudinal axial direction of the mesh structure; and
at least one embolization means for placement in the aneurysm, the embolization means formed by one of a plastically deformable wire or a liquid.
